(19) United States
(12) Patent Application Publication
(10) Pub. No.: US 2020/0188044 A1
(43) Pub. Date: Jun. 18, 2020

(54) Guidance of Robotically Controlled Instruments Along Paths Defined with Reference to Auxiliary Instruments

(71) Applicant: TransEnterix Surgical, Inc., Morrisville, NC (US)

(72) Inventors: Matthew Robert Penny, Holly Springs, NC (US); Kevin Andrew Hufford, Cary, NC (US); Mohan Nathan, Raleigh, NC (US); Glenn Warren, Raleigh, NC (US)

(21) Appl. No.: 16/733,147

(22) Filed: Jan. 2, 2020

Related U.S. Application Data
(63) Continuation of application No. 16/010,388, filed on Jun. 15, 2018.
(60) Provisional application No. 62/787,250, filed on Dec. 31, 2018.

(51) Int. Cl. A61B 34/30; A61B 34/00; A61B 90/00

(52) U.S. Cl. CPC A61B 34/30; A61B 34/76; A61B 2034/301; A61B 90/361; A61B 90/37

(57) ABSTRACT

A robot-assisted surgical system includes a robotic manipulator configured for robotic positioning of a surgical instrument in a body cavity, a surgical instrument positionable in an operative site in the body cavity and at least one path-defining instrument insertable into a natural body orifice. The system is configured to determine a position of the path-defining instrument. A target resection path for the surgical instrument may be determined based on the determined position. The path-defining instrument may be a bougie or colpotomy ring.

### BACKGROUND

There are various types of surgical robotic systems on the market or under development. Some surgical robotic systems use a plurality of robotic arms. Each arm carries a surgical instrument, or the camera used to capture images from within the body for display on a monitor. Other surgical robotic systems use a single arm that carries a plurality of instruments and a camera that extend into the body via a single incision. Each of these types of robotic systems uses motors to position and/or orient the camera and instruments and to, where applicable, actuate the instruments. Typical configurations allow two or three instruments and the camera to be supported and manipulated by the system. Input to the system is generated based on input from a surgeon positioned at a master console, typically using input devices such as input handles and a foot pedal. Motion and actuation of the surgical instruments and the camera is controlled based on the user input. The image captured by the camera is shown on a display at the surgeon console. The console may be located patient-side, within the sterile field, or outside of the sterile field.

Although the inventions described herein may be used on a variety of robotic surgical systems, the embodiments will be described with reference to a system of the type shown in FIG. 1. In the illustrated system, a surgeon console 12 has two input devices such as handles 17, 18 that the surgeon selectively assigns to two of the robotic manipulators 13, 14, 15, allowing surgeon control of two of the surgical instruments 10a, 10b, and 10c disposed at the working site at any given time. To control a third one of the instruments disposed at the working site, one of the two handles 17, 18 is operatively disengaged from one of the initial two instruments and then operatively paired with the third instrument. A fourth robotic manipulator, not shown in FIG. 1, may supports and maneuvers an additional instrument.

One of the instruments 10a, 10b, 10c is a laparoscopic camera that captures images for display on a display 23 at the surgeon console 12. The camera may be moved by its corresponding robotic manipulator using input from an eye tracker 21.

The input devices at the console may be equipped to provide the surgeon with tactile feedback so that the surgeon can feel on the input devices 17, 18 the forces exerted by the instruments on the patient's tissues.

A control unit 30 is operationally connected to the robotic arms and to the user interface. The control unit receives user input from the input devices corresponding to the desired movement of the surgical instruments, and the robotic arms are caused to manipulate the surgical instruments accordingly.

New opportunities for control of the surgical instruments arise when the system is paired with other surgical implements such as a colpotomy ring, stomach bougie, stent or catheter. This application describes embodiments where the surgical robotic system is capable of identifying and responding to other surgical implements, intraoperatively.

### BRIEF DESCRIPTION OF THE DRAWINGS

FIG. 1 schematically illustrates elements of a surgical robotic system of a type that may be adapted for use with the disclosed invention.

FIGS. 2A-2C show a sequence of drawings that schematically depict a surgical method in which a stomach pouch is created along a bougie positioned to extend from the esophagus to the pylorus;

FIG. 3 schematically depicts use of a uterine manipulator with colpotomy ring.

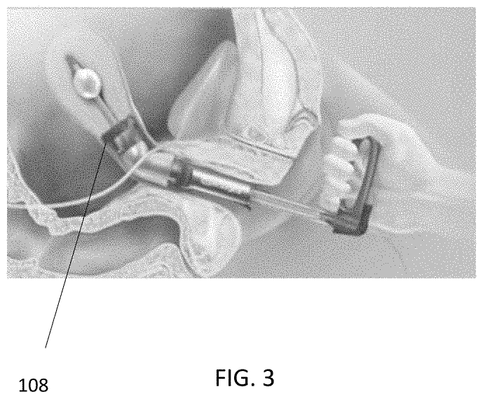

### DETAILED DESCRIPTION

This application describes modes and methods of operation for a surgical robotic system according to which the system may identify and respond to other, intraoperatively. While the modes and methods are not limited to any specific types of surgical procedures, the embodiments describe operation of the system in which a colpotomy ring/cup is used during a total laparoscopic hysterectomy, and one in which and a stomach bougie is used for a sleeve gastrectomy.

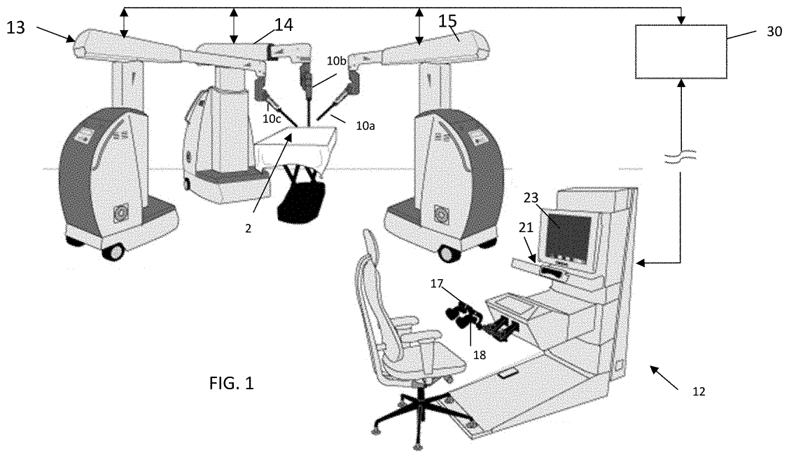

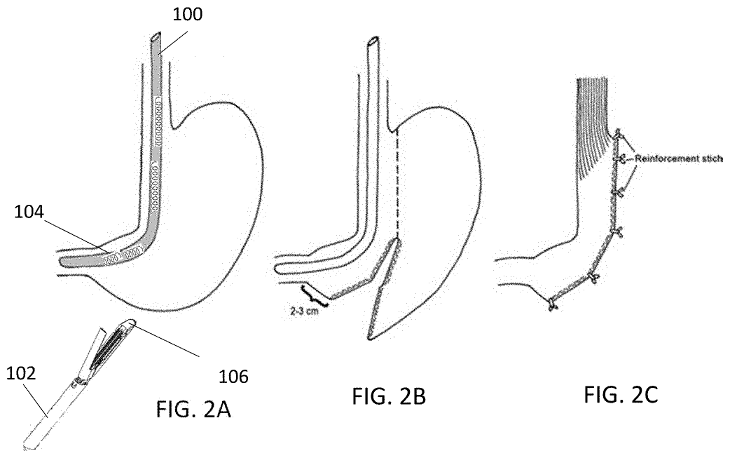

Referring to FIG. 2A, in a first embodiment, a surgical robot system (FIG. 1) which robotically manipulates a surgical instrument is used together with a stomach bougie 100 during a sleeve gastrectomy. The robotically moveable surgical instrument is preferably the surgical stapler 102 to be used to resect and fasten the stomach tissue to form the pouch. During sleeve gastrectomy, the stomach pouch to be formed is defined by positioning the bougie extending through the stomach, from the esophagus to the pylorus. The surgeon typically feels for the bougie with an instrument positioned at the stomach, such as the stapler that will be used to form the pouch, prior to beginning the staple line. The size of the finished sleeve is dictated by how close the surgeon gets the stapler to the bougie, the size of the bougie and whether or not the surgeon over-sews the staple line. The distance between the stapler and the bougie is defined only by the surgeon's estimation.

In the FIG. 2A embodiment, the system is configured to estimate the relative positions of the bougie and the stapler and to communicate that information to the surgeon and/or to control the surgeon's use of the stapler depending on whether the stapler is in the proper position and orientation to form the staple line and cut at the desired distance (or within the desired distance range) from the bougie. In one embodiment, there is communication between one or more elements 104, 106 on the stapler 102 end effector and bougie 100 that allow the system to help identify and confirm the staple line for the surgeon. A specific implementation of this embodiment would embed one or more inductive antennas 104 in the bougie 100. The antennas have a circulating current that fluctuates depending on the proximity of the stapler end effector. In another embodiment, the bougie may include one or more inductive proximity sensors that determine when the metal of the stapler is within a predetermined distance from the coil.

The surgeon could pre-define a desired sleeve width and the system would help to confirm the position of the stapler with respect to the bougie prior to firing. This confirmation of position could also include a haptic component that causes the use input device to apply force to the surgeon's hand. This force could restrain movement of the user input handle to restrict motion of the stapler along the path, or cause the surgeon to haptically feel as if the instrument is attracted to the path (like a magnet), thus compelling the surgeon to move the handle so as to guide the instrument along that path.

In a modified version of the first embodiment, a memory of the system stores a computer program that includes a computer vision algorithm. A controller executes the computer vision algorithm to analyze endoscopic image data, 3D endoscopic image data or structured light system image data to detect shape characteristics of the stomach as shaped by the bougie. The algorithm is used to determine the location of the bougie based on topographical variations in the imaged region or, if the bougie is illuminated, light variations. The system can generate an overlay on the image display identifying the location of the bougie or a margin of predetermined distance from the detected longitudinal edge of the bougie. The surgeon can the guide the stapler to a target cut/staple pathway based on the region defined by the bougie. Alternatively, the system can generate a haptic boundary as described above, allowing the surgeon to advance the stapler along the haptic boundary to complete the stapling and cutting steps. Additionally, or as an alternative, the system may be configured so that the user cannot fire the stapler except when the stapler is an appropriate position to create the pouch, such as a predetermined distance from the bougie, oriented along the target staple pathway, etc.

A second embodiment would enable the use of a surgical robotic system with a colpotomy ring and uterine manipulator. During a hysterectomy, it is necessary to cut the vaginal cuff circumferentially to detach the uterus from the vagina. As with the bougie, the colpotomy ring is not readily identifiable when inserted into the patient due to the layer of tissue between the device and the robotically controlled surgical instruments.

Much like the bougie example, the second embodiment would enable communication between the uterine manipulator, specifically the colpotomy ring 108, and the surgical system such that the surgical system could identify the location of the colpotomy ring and the instrument proximity to the ring. As in the bougie example, control of the user input devices can be used to deliver haptic feedback that causes the surgeon to feel as if the instruments are haptically attracted to a path defined by the circumference of the ring. Electrosurgical devices used for the procedure may be set up so that their energy-delivery features are enabled when within the ring proximity, as a means to prevent undesired tissue damage.

These modes of operation could be turned on or off by a surgeon using input at the surgeon console or enabled via procedural anticipation based on observed steps motions (using kinematics or computer vision techniques) being carried out during the procedure.

These embodiments provide a number of advantages over existing technology, including:
Path definition using other intraoperative surgical implements.
Operative modes for a surgical robot based on paths defined by the location of other surgical implements

Described concepts that are particularly unique include:
a robotic surgical system having a mode of operation that enables the system to provide boundaries or paths based on the location of other intraoperative surgical equipment.
boundaries and paths that can be felt by a user via haptic constraints, attractions or repulsions.
operative modes that enable the use of features such as energy delivery features or staple/fastener/suture application features when near identified paths, objects or boundaries
operative modes that disable the use of such features when near identified paths, objects or boundaries.

Concepts described in U.S. application Ser. No. 16/237,418, “Use of Eye Tracking for Tool Identification and Assignment in a Robotic Surgical System” (Ref: TRX-14210) and U.S. application Ser. No. 16/237,444 “System and Method for Controlling a Robotic Surgical System Based on Identified Structures” (Ref: TRX-14410), and U.S. Provisional 62/787,250, entitled “Instrument Path Guidance Using Visualization and Fluorescence” (Ref: TRX-14000) may be combined with those discussed in the present applications.

All patents and applications referenced herein, including for purposes of priority, are incorporated herein by reference.

What is claimed is:

1. A robot-assisted surgical system comprising:
a robotic manipulator configured for robotic positioning of a surgical instrument in a body cavity,
a surgical instrument positionable in an operative site in the body cavity;
at least one path-defining instrument insertable into a natural body orifice, the surgical instrument in wireless electronic communication with the surgical instrument at least one processor and at least one memory, the at least one memory storing instructions executable by said at least one processor to:
receive user input in response to movement of the input device by a user
cause the manipulator to move the first surgical instrument in response to the user input,
receive signals from at least one of the surgical instrument and the path-defining instrument, and, based on the received signals, determining a target resection path for the surgical instrument.

2. The system of claim 1, wherein the instructions are executable to haptically constrain movement of the user input device to restrict movement of the surgical instrument to the target resection path.

3. The system of claim 1, wherein the instructions are executable to generate a visual overlay on an image display of the body cavity, the visual overlay depicting a boundary of the target resection path.

4. A robot-assisted surgical system comprising:
a robotic manipulator configured for robotic positioning of a surgical instrument in a body cavity,
a surgical instrument positionable in an operative site in the body cavity;
at least one path-defining instrument insertable into a natural body orifice;
a camera for generating an image of the body cavity;
at least one processor and at least one memory, the at least one memory storing instructions executable by said at least one processor to:
receive user input in response to movement of the input device by a user
cause the manipulator to move the first surgical instrument in response to the user input,
detect using image processing the position of at least an edge the path-defining instrument within the body cavity and, based on the determined position, determining a target resection path for the surgical instrument.

5. The system of claim 4, wherein the instructions are executable to haptically constrain movement of the user input device to restrict movement of the surgical instrument to the target resection path.

6. The system of claim 4, wherein the instructions are executable to generate a visual overlay on an image display of the body cavity, the visual overlay depicting a boundary of the target resection path.

7. A surgical system including:
a robotically controlled surgical instrument;
a path-defining instrument,
the system configured to define a target path or position for the surgical instrument based on the position or location of the path-defining instrument within the patient.

8. The system of claim 7, where the system uses non-contact methods to define the distance between surgical instrument and the path-defining instrument.

9. The system of claim 8, wherein the non-contact methods include antennas or other near field communication equipment.

10. A system of claim 8, where the system prevents a function of the surgical instrument when it is near a defined path, object or boundary.

11. The system of claim 8, wherein the system enables a function of the surgical instrument when it is near a defined path, object or boundary.

12. The system of claim 10, wherein the function is energy delivery or deliver of a staple or other fastener.

13. The system of claim 8, wherein the system causes the surgeon to “feel” the defined path, object or boundary via haptics provided to a surgeon input device.

14. The system of claim 1, wherein the path-defining instrument is a bougie.

15. The system of claim 1, wherein the path-defining instrument is a colpotomy ring.

16. The system of claim 7, wherein:
the system includes at least one processor and at least one memory, the at least one memory storing instructions executable by said at least one processor to:
detect using image processing the position of at least an edge the path-defining instrument within the body cavity and, based on the determined position, determining a target path or position for the surgical instrument.

17. The system of claim 7, wherein:
the system includes at least one processor and at least one memory, the at least one memory storing instructions executable by said at least one processor to:
receive signals from at least one of the surgical instrument and the path-defining instrument, and, based on the received signals, determining a position of the path-defining instrument and determining a target resection path for the surgical instrument.
